(19) United States
(12) Patent Application Publication
(10) Pub. No.: US 2005/0019268 A1
(43) Pub. Date: Jan. 27, 2005

(54) Spray containing ubiquinone Qn

(75) Inventor: Franz Enzmann, Bad Homburg (DE)

Correspondence Address: JACOBSON HOLMAN PLLC, 400 SEVENTH STREET N.W., SUITE 600, WASHINGTON, DC, 20004, US

(73) Assignee: MSE Pharmazeutika GmbH

(21) Appl. No.: 10/624,888

(22) Filed: Jul. 23, 2003

Related U.S. Application Data
(63) Continuation of application No. 09/890,277, filed on Aug. 10, 2001, now abandoned.

(30) Foreign Application Priority Data
Feb. 11, 1999 (DE) 199 05 880.6

(51) Int. Cl. A61L 9/04; A61K 31/12

(52) U.S. Cl. 424/45; 514/690

(57) ABSTRACT

Ubiquinone Qₙ is employed as a spray together with usual auxiliaries.

Ubiquinones are prenylated quinones which are wide-spread in the animal and vegetable kingdoms. They are derivatives of 2,3-dimethoxy-5-methyl-1,4-benzoquinone having linearly linked isoprene units in the 6-position. Depending on the number of isoprene units, the ubiquinones are designated as Q-1, Q-2, Q-3 etc. In most mammals including humans, Q-10 (2,3-dimethoxy-5-methyl-6-deca-prenyl-1,4-benzoquinone) is prevailing. Ubiquinones serve as electron carriers in the respiratory chain, and they participate in the cyclic oxidation and reduction of substrates in the citric acid cycle. Ubiquinones Qₙ represent a precondition of the energy supply to all cells. The oxidative stress which arises, inter alia, from a high oxygen consumption causes damage to the membranes of mitochondria and cells which result in acute or degenerative disorders of the nervous system. The nervous system has a very high energy demand for the signal transduction by membrane potential build-up, ion-channel control, as well as by neuropeptide and neurotransmitter vesicle formation.

Ubiquinone Q-10 (also referred to as coenzyme Q-10) has previously been used in the therapy of heart diseases.

The subject matter of the invention is a spray containing ubiquinone Qₙ or ubiquinone Qₙ precursors together with usual auxiliaries.

The term “ubiquinone Qₙ precursors” refers to compounds which are converted to ubiquinone Qₙ in the body. These include, on the one hand, the ubihydroquinones, which are in an equilibrium with the ubiquinones, as well as simple esters of the ubihydroquinones with short-chained carboxylic acids having from 1 to 10 carbon atoms, for example, acetate, propionate or butyrate esters. These precursors are converted to the corresponding ubiquinones after the application thereof.

Ubiquinone Q-10 is preferably used because this is the main ubiquinone in humans.

The spray according to the invention is preferably an oral or nasal spray, so that the administration of ubiquinone Qₙ or ubiquinone Qₙ precursors can be effected on an inhalative or intranasal route. The spray according to the invention is useful, in particular, for the treatment of pain conditions as encountered in migraine and neuropathy, in neural disorders, such as depressions, psychoses, lack of concentration, and in neurodegenerative diseases, such as Alzheimer's, Parkinson's, Huntington's, multiple sclerosis and cerebral paresis. The application as an oral or nasal spray seems to accelerate the transport of ubiquinone Qₙ or its precursors through the blood-brain barrier. It was found that the oral administration of from 600 to 800 mg of Q-10 in the form of capsules exhibited virtually no effect on migraine, whereas a nasal formulation could effectively alleviate pain from migraine and tension headaches already at a low amount. Thus, doses of about 20 mg were sufficient for a significant alleviation of pain. In principle, however, doses of as high as 1000 mg can be employed.

Ubiquinones are lipophilic substances which are virtually insoluble in water. However, a particularly high effectiveness was found when the ubiquinone Qₙ or its precursors are in aqueous dispersion. Aqueous colloidal dispersions are particularly preferred. The preparation of the corresponding dispersions is described in WO 95/05164 and in the related DE-A-43 27 063.

1-5. (canceled)

6. A spray containing ubiquinone Q-10 in an aqueous colloidal dispersion.

7. The spray according to claim 6, characterized by being an oral or nasal spray.

8. A method of using the spray according to claim 6 comprising administering a dose of the spray to a person in need thereof, by spraying orally or nasally, for the treatment of pain conditions from neural disorders, wherein the ubiquinone Q-10 is present in amount of 20-1000 mg/dose.

9. A method of using the spray according to claim 6 comprising administering the spray to a person in need thereof, by spraying orally or nasally, for the treatment of migraine or neuropathy.

10. A method of using the spray according to claim 6 comprising administering the spray to a person in need thereof, by spraying orally or nasally, for the treatment of depression, psychosis, or lack of concentration.

11. A method of using the spray according to claim 6 comprising administering a dose of the spray to a person in need thereof, by spraying orally or nasally, for the treatment of Alzheimer's, Parkinson's, Huntington's, multiple sclerosis, or cerebral palsy, wherein ubiquinone Q-10 is present at an amount of 20-1000 mg/dose.
